(19) United States
(12) Patent Application Publication
(10) Pub. No.: US 2024/0325709 A1
(43) Pub. Date: Oct. 3, 2024

(54) STRAIGHT CANULA GROOVE HEMOSHIELD CONNECTOR

(71) Applicant: CareFusion 303, Inc., San Diego, CA (US)

(72) Inventors: Seyed Alireza SALEHI BOROJERDI, Aliso Viejo, CA (US); Amarsinh Deeliprao JADHAV, Bangalore (IN); Mohammed Mehtab KHAN, Bengaluru (IN); Shelby Ann BIERITZ, Durham, NC (US)

(21) Appl. No.: 18/595,990

(22) Filed: Mar. 5, 2024

Related U.S. Application Data
(60) Provisional application No. 63/455,211, filed on Mar. 28, 2023.

(51) Int. Cl. A61M 39/10; A61B 5/15

(52) U.S. Cl. CPC A61M 39/10; A61B 5/150992; A61M 2039/1083; A61M 2039/1088; A61M 2207/10

(57) ABSTRACT

A fluid connector device for a blood collection system is disclosed. The fluid connector device may have passage for regulating movement of a fluid therethrough, including a flow restriction device having an exterior flow channel to regulate a fluid flow through the device. The fluid connector device can include a first connector having an internal surface defining an inner lumen, a second connector coupled to an end of the first connector, and a flow restrictor insert contained within the lumen of the first and second connector.

### CROSS REFERENCE TO RELATED APPLICATION

This application claims the benefit of priority under 35 U.S.C. § 119 to U.S. Provisional Patent Application Ser. No. 63/455,211, entitled “Straight Canula Groove Hemoshield Connector”, filed on Mar. 28, 2023, the entire contents of which is incorporated herein by reference.

### TECHNICAL FIELD

The present disclosure generally relates to blood draw and administration of parenteral fluids to a patient, and particularly to improve the manufacturability of a blood flow channel for a Hemoshield Connector.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used as an internal vacuum or a vacuum tube. The blood collection container may also be a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container.

The description provided in the background section should not be assumed to be prior art merely because it is mentioned in or associated with the background section. The background section may include information that describes one or more aspects of the subject technology.

### SUMMARY

The present disclosure provides devices and accessories for reducing hemolysis that can include features for restricting and regulating a fluid flow therethrough.

In some instances, the present disclosure provides flow restriction devices that can regulate a fluid flow moving through the device in one or more direction, such as a first fluid flow moving in a direction away from a patient and a second fluid flow moving in a direction toward the patient.

Embodiments of the present disclosure provide a fluid connector device, comprising a first connector, a second connector that combines with the first connector, forming a housing, and a flow restrictor insert with an exterior flow channel, wherein the insert is fitted inside the housing.

In some embodiments, the exterior flow channel comprises a groove extending from a proximal end of the flow restrictor insert to a distal end of flow restrictor insert. In some embodiments, the groove is a semi-parabolic cylinder. In some embodiments, the first connector is a female luer. In some embodiments, the second connector is a male luer.

In some embodiments, the exterior flow channel has a diameter of about 0.015 in., about 0.020 in., about 0.025 in., about 0.030 in., about 0.035 in., or about 0.040 in. In some embodiments, the exterior flow channel has a length of about 0.70 in., about 0.80 in., about 0.90 in., about 1.0 in., about 1.10 in., about 1.20 in., or about 1.30 in. In some embodiments, the flow restrictor insert has a maximum diameter of about 0.1 in., about 0.2 in., about 0.3 in., about 0.4 in., about 0.5 in., about 0.6 in., or about 0.7 in. In some embodiments, the first connector has an inner diameter of about 0.1 in., about 0.2 in., about 0.3 in., about 0.4 in., about 0.5 in., about 0.6 in., or about 0.7 in. In some embodiments, the second connector has an inner diameter of about 0.1 in., about 0.2 in., about 0.3 in., about 0.4 in., about 0.5 in., about 0.6 in., or about 0.7 in.

In some embodiments, a fluid flows through the exterior flow channel from the first connector towards the second connector. In some embodiments, the fluid is blood. In some embodiments, the flow restrictor insert reduces hemolysis during blood draw. In some embodiments, the fluid connector device is configured to couple to a catheter assembly. In some embodiments, the flow restrictor insert is contained within the first connector and the second connector. In some embodiments, the flow restrictor insert is a solid cylinder with the exterior flow channel carved out of an exterior surface of the flow restrictor insert.

Embodiments of the present disclosure provide a blood collection system comprising a blood collection device, and a fluid connector device fluidly coupled to the blood collection device, the fluid connector device comprising a first connector, a second connector that combines with the first connector, forming a housing, and a flow restrictor insert with an exterior flow channel, wherein the insert is fitted inside the housing.

Embodiments of the present disclosure provide a method for manufacturing a fluid connector device comprising obtaining a first connector, inserting a flow restrictor insert with an exterior flow channel inside the first connector, obtaining a second connector and coupling the first connector to the second connector forming a housing; wherein the flow restrictor insert is press fitted inside the housing. In some embodiments, the method further comprises forming the flow restrictor insert into a solid cylinder with a groove carved out of the external surface. In some embodiments, the method further comprises ultrasonically welding the first connector to the second connector.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1A illustrates a vascular access device including a peripheral intravenous catheter (PIVC) assembly that includes a fluid connector device, in accordance with some embodiments of the present disclosure.

FIG. 1B illustrates a perspective exploded view of components of the fluid connector device of FIG. 1A, in accordance with some embodiments of the present disclosure.

FIG. 2 illustrates an exploded perspective view of the fluid connector device of FIG. 1A, in accordance with some embodiments of the present disclosure.

FIG. 3 illustrates a cross-sectional view of the fluid connector device of FIG. 1A, in accordance with some embodiments of the present disclosure.

FIG. 4 illustrates a cross-sectional view of the fluid connector device of FIG. 1A, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing systems and methods to address hemolysis in PIVC blood draw with a hemolysis reduction accessory (also referred to herein as a fluid connector device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a fluid connector device) that regulates the overall flow rate of the entire fluid path as blood cells travel through. The fluid connector device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the fluid connector device and the blood collection device together. As such, this fluid connector device can be either for single blood draw or stay inline throughout indwell.

In accordance with various embodiments of the present disclosure, the fluid connector device may include a distal connector configured to couple to the catheter assembly. The distal connector may have a proximal end including a first connection portion, and a distal end including a second connection portion. The first connection portion may have an inner surface defining a lumen therethrough, and the second connection portion may have an inner surface defining a lumen therethrough. The distal connector may be in the form of a male luer connector or another suitable connector, which may be coupled with a female luer portion of the needleless connector which may be fluidly coupled to the catheter via the extension tubing.

In some embodiments, the fluid connector device may further include a proximal connector coupled to the proximal end of the distal connector. The proximal connector may be configured to couple to fluid collection device (e.g., a blood collection device). For example, the proximal connector may be integrated with the blood collection device or monolithically formed with the blood collection device as a single unit. As another example, the proximal connector may be in the form of a female luer connector or another suitable connector, which may be coupled with a male luer portion of the blood collection device. The proximal connector may have a proximal end, a distal end, and an internal surface defining a lumen extending therethrough for coupling to the male luer portion of the blood collection device.

In accordance with various embodiments of the present disclosure, the fluid connector device may further include an insert (flow restrictor) fitted in the lumen of the first connection portion and interposed between the proximal connector and the distal connector. As depicted, the insert may have an outer surface, a proximal end, and a distal end. The insert may further include a flow channel, or a plurality of flow channels, extending longitudinally from the proximal end to the distal end of the insert for fluidly communicating the lumen of the first connection portion of the distal connector with the lumen of the proximal connector.

In some embodiments, the flow restrictor channel may be in the form of a micro-channel fluid pathway through which fluid (e.g., blood) flows from the distal connector to the proximal connector for collection in the fluid collection device. The flow restrictor channel in the form of a micro-channel along which fluid (e.g., blood) may flow from the distal connector into the proximal connector for collection into the fluid collection device, may have a diameter in the range of 0.015 inches to 0.15 inches, in some instances 0.02 inches to 0.1 inches, in other instances 0.04 inches to 0.08 inches, in other instances approximately 0.06 inches. Accordingly, during blood collection or withdrawal from the patient, the blood may flow into the blood collection device via the micro-channel defined in the blood flow channel having minimal diameter. The fluid connector device of the various embodiments described herein is advantageous over currently existing blood collection systems. For example, during blood draw with currently existing blood draw devices, blood cells may experience wall shear stress as they flow from the distal end to the proximal end of the blood collection systems. As previously described above, wall shear stress on blood cells is considered a major source of mechanical damage to blood cells causing hemolysis of the blood cells. The micro-channel defined in the blood draw channel having minimal diameter may facilitate increased flow resistance within the vascular access system to distribute the pressure differential and reduce shear stress experienced by the red blood cells of the blood. For example, the minimized diameter of the blood draw channel may provide increased resistance to flow of the blood and thereby decrease blood flow rate within the flow connector device. Since the decreased blood flow rate causes a reduction in shear stress experienced by the red blood cells of the blood, a risk of hemolysis during blood collection may advantageously be reduced.

The micro-channel is very small in diameter and comparatively lengthier, which causes manufacturing issues. In accordance with various embodiments of the present disclosure, the insert may have a flow channel on the exterior surface of the insert, which improves manufacturability of the micro-channel.

Where the medical fluid is blood being withdrawn or collected from a patient, the medical fluid may be a blood sample, and the fluid collection device may be a blood collection device. In some embodiments, the blood collection device may be a luer lock access device (LLAD).

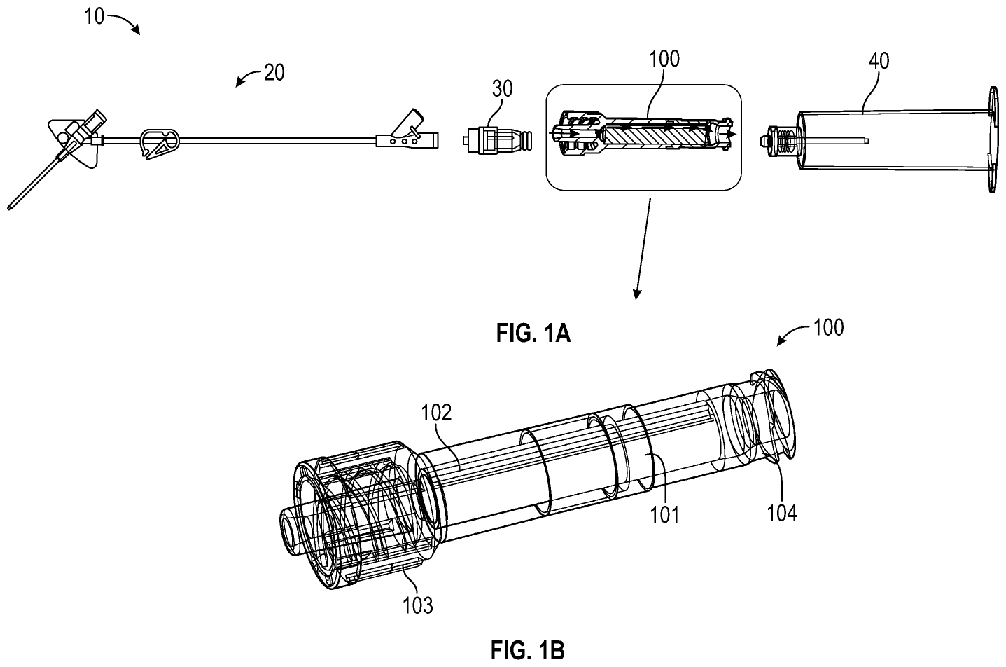

FIG. 1A illustrates a vascular access device including a peripheral intravenous catheter (PIVC) assembly that includes a fluid connector device 100, in accordance with some embodiments of the present disclosure. The fluid connector device 100 may be configured to reduce a likelihood of hemolysis during blood collection using a vascular access device. In some embodiments, the vascular access device may include a catheter assembly (e.g., a PIVC). In some embodiments, the fluid connector device 100 may include a distal end, which may include a body or distal connector configured to couple to the catheter assembly. The distal connector may include a male luer connector, or another suitable connector. In some embodiments, the catheter assembly may include a catheter hub, which may include a distal end, a proximal end, and a lumen extending through the distal end and the proximal end. The catheter assembly may further include a catheter, which may be secured within the catheter hub and may extend distally from the distal end of the catheter hub. In some embodiments, the catheter may be a peripheral intravenous catheter (PIVC).

FIG. 1A illustrates a collection system 10, in accordance with some aspects of the present disclosure. The collection system 10 is designed to collect fluid (e.g., blood) from a patient. Accordingly, the collection system 10 may be referred to as a blood collection system. The collection system 10 can take the form of a vascular access device with a PIVC assembly. As shown, the collection system 10 include a catheter assembly 20. In some embodiments, the catheter assembly 20 may include or correspond to any suitable catheter assembly. In some embodiments, the catheter assembly 20 may be integrated with and include an extension tube, which may extend from and be integrated with a side port of the catheter hub. A non-limiting example of an integrated catheter assembly is the BD NEXIVA™ Closed IV Catheter system, available from Becton Dickinson and Company. In some embodiments, a proximal end of the extension tube may be coupled to an adapter, such as, for example, a Y-adapter or single port luer adapter.

Also, the collection system 10 may include a connector 30 that connects to the catheter assembly 20. In order to collect fluid (e.g., blood) from a patient, the collection system 10 further includes a collector 40. When the collected fluid is blood from a patient, the collector 40 may be referred to as a blood collector.

Additionally, the collection system 10 includes a fluid connector device 100, in accordance with some aspects of the present disclosure. The fluid connector device 100 may be configured to reduce a likelihood of hemolysis during blood collection using the collection system 10. In some embodiments, the fluid connector device 100 may include a distal end, which may include a body or distal connector configured to couple to the catheter assembly 20 by way of the connector 30. The connector 30 may include a male luer connector, or another suitable connector.

In some embodiments, the distal connector of fluid connector device 100 may be configured to couple to the Y-adapter of the catheter assembly 20 without the use of the connector 30. In some embodiments, the catheter assembly 20 may be non-integrated and may not include the extension tube. In these and other embodiments, the fluid connector device 100 may be configured to couple to the proximal end of the catheter hub or another suitable portion of the catheter assembly. In some embodiments, the catheter assembly 20 may be coupled to a removable extension tube. In some embodiments, the fluid connector device 100 may be coupled directly to the catheter adapter, eliminating the extension tube and providing a compact catheter system.

FIG. 1B illustrates a perspective view of the fluid connector device 100 shown in FIG. 1A, in accordance with some aspects of the present disclosure. The fluid connector device 100 may include a female luer 103 on the proximal end, a flow restrictor insert 101, and a male luer 104 on the distal end. The fluid restrictor insert may be altered based on the application of the fluid connector device and has a uniform channel, which supports effective flushing and reduces risk of hemolysis.

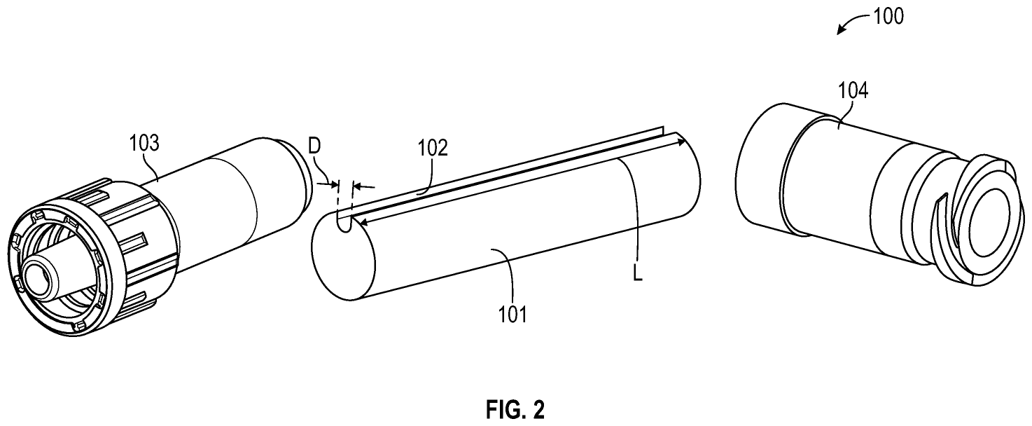

With reference to FIG. 2, the fluid connector device 100 may include a three-part design. In some embodiments, the fluid connector device 100 includes a first connector 103 and a second connector 104 that combines with the first connector 103, forming a housing. In some embodiments, the fluid connector device 100 can include a first connector 103 having an internal surface defining an inner lumen, a second connector 104 coupled to an end of the first connector 103, and a flow restrictor insert 101 contained within the lumen of the first and second connector. In some embodiments, the fluid connector device 100 includes a flow restrictor insert 101 with an exterior flow channel 102. In some embodiments, the insert is fitted (i.e., press fitted) inside the housing formed by the coupling of the first connector 103 with the second connector 104. In some embodiments, the first connector 103 is a female luer and the second connector 104 is a male luer. In the fully assembled fluid connector device, the flow restrictor insert 101 is contained within the male and female luers.

In some embodiments, the exterior flow channel 102 comprises a groove extending from a proximal end of the flow restrictor insert 101 to a distal end of flow restrictor insert 101. In some embodiments, the exterior flow channel 102 is a semi-parabolic cylinder. In some embodiments, the exterior flow channel 102 is a semi-cylinder.

In some embodiments, the exterior flow channel 102 has a diameter D of about 0.015 in., about 0.020 in., about 0.025 in., about 0.030 in., about 0.035 in., or about 0.040 in. In some embodiments, the exterior flow channel 102 has a length L of about 0.70 in., about 0.80 in., about 0.90 in., about 1.0 in., about 1.10 in., about 1.20 in., or about 1.30 in.

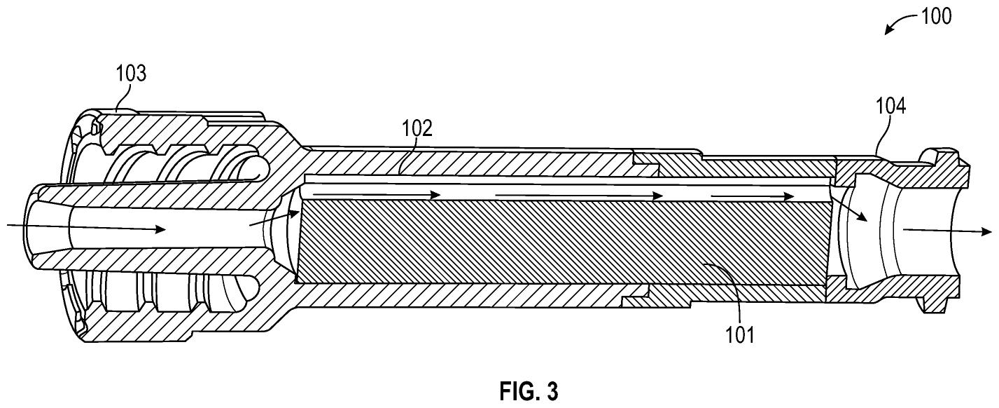

With reference to FIG. 3, this exterior flow channel 102 may act as a blood flow path for a fluid connector device 100. A fluid (i.e., blood) may flow through the exterior flow channel 102 from the first connector 103 towards the second connector 104. The flow restrictor insert 101 may be easy to manufacture as the groove is placed on the external surface of the flow restrictor insert 101.

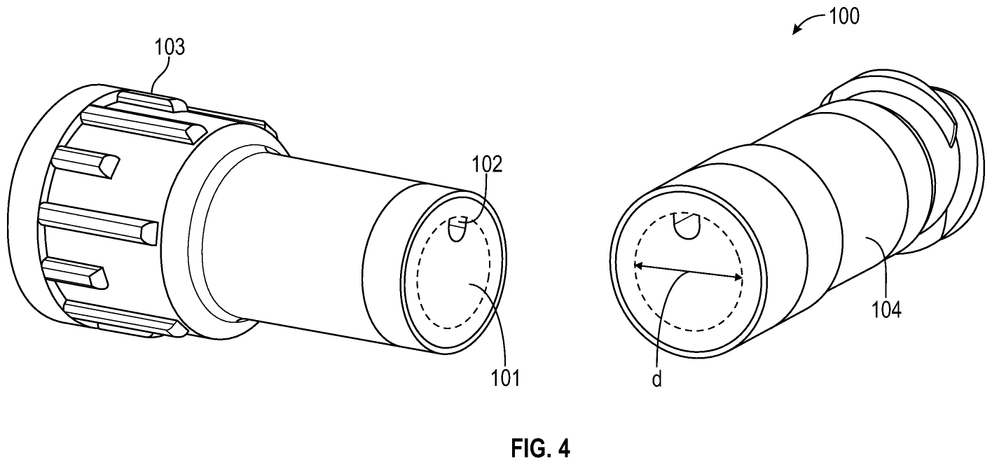

With reference to FIG. 4, the flow restrictor insert 101 may be contained within the lumen defined by the first connector 103 and second connector 104. The flow restrictor insert 101 may comprise a maximum diameter d based on the size of the first connector 103 and second connector 104, so that the flow restrictor insert 101 may be press fitted inside the lumen defined by the first connector 103 and second connector 104. In some embodiments, the flow restrictor insert has a diameter d of about 0.1 in., about 0.2 in., about 0.3 in., about 0.4 in., about 0.5 in., about 0.6 in., or about 0.7 in. 9. In some embodiments, the first connector 103 has an inner diameter of about 0.1 in., about 0.2 in., about 0.3 in., about 0.4 in., about 0.5 in., about 0.6 in., or about 0.7 in. the second connector 104 has an inner diameter of about 0.1 in., about 0.2 in., about 0.3 in., about 0.4 in., about 0.5 in., about 0.6 in., or about 0.7 in.

In some embodiments, the disclosure relates to a blood collection system comprising a blood collection device and a fluid connector device 100 fluidly coupled to the blood collection device. In some embodiments, the fluid connector device 100 comprises a first connector 103 and a second connector 104 that combines with the first connector 103, forming a housing. In some embodiments, the blood collection device comprises a flow restrictor insert 101 with an exterior flow channel 102, wherein the insert 101 is fitted inside the housing.

In some embodiments, the disclosure relates to a method for manufacturing a fluid connector device 100. The method may include obtaining a first connector 103, inserting a flow restrictor insert 101 with an exterior flow channel 102 inside the first connector 103, obtaining a second connector 104, and coupling the first connector 103 to the second connector 104 forming a housing. In some embodiments, the flow restrictor insert 101 is press fitted inside the housing. In some embodiments, the method includes forming the flow restrictor insert 101 into a solid cylinder with a groove carved out of the external surface to form flow channel 102. In some embodiments, flow channel 102 extends from a proximal end of the flow restrictor insert 101 to a distal end of the flow restrictor insert 101. In some embodiments, the flow channel 102 could be a semi-parabolic cylinder. In some embodiments, the flow channel 102 could be a semi-cylinder. In some embodiments, the method further comprises ultrasonically welding the first connector 103 to the second connector 104.

The fluid connector devices and associated blood collection systems of the various embodiments described herein additionally provide further advantages over currently existing blood collection systems. For example, add-on fluid connector devices described herein allow for hemolysis-reduction function to be integrated for PIVC blood draw. Further, the fluid connector devices described herein are compatible with PIVC placement and allow for seamless blood draw at insertion. Ion some embodiments, the fluid connector devices have the potential to stay inline throughout PIVC indwell for multiple blood draws. Additionally, since the fluid connector devices are an add-on which can be easily incorporated without any changes to existing PIVC, there is minimal impact to clinical setting and operations.

An optimized fluid pathway, also referred to herein as a first fluid pathway or flowpath or micro-channel, can be configured for providing a restricted flow rate for reducing hemolysis, and can have features including, but not limited to a tubular fluid pathway, a cannula, a lumen, a continuous non-linear channel, a groove, a fluid channel, and the like.

The fluid pathway can have a length that is selected based on one or more of the following: a gauge of a particular catheter, a particular catheter assembly configuration, or a clinical setup. In some embodiments, the optimized fluid pathway may include a length L from the female luer adapter 103 to the male luer adapter 104. In some embodiments, the optimized fluid pathway may include an inner diameter D.

Fluid flow in a tubular fluid pathway therethrough can be analyzed using Poiseuille's equation:

$$Q = \frac{π {D}^{4} Δ P}{128 μL} = \frac{Δ P}{{R}_{f}}$$

where ΔP is a change in pressure gradient across the length of the fluid pathway, D and L are the inner diameter and length, respectively, of the fluid pathway, μ is the viscosity of a fluid, and

$${R}_{f} = \frac{128 μL}{π {D}^{4}}$$

is the fluid resistance. Since μ is the viscosity of the fluid and not part of the extension tube geometry, a geometric factor G_{f} is defined such that R_{f} (the fluid resistance) is

$${R}_{f} = \frac{128 μL}{π} {G}_{f} , where {G}_{f} = \frac{L}{{D}^{4}} .$$

In some embodiments, the optimized fluid pathway may have multiple sections with lengths (L1, L2, L3) and inner diameters of (D1, D2, D3), the geometric factor is then:

$${G}_{f} = {\int }_{0}^{L} \frac{dl}{{D ( l )}^{4}}$$

In some embodiments, the optimized fluid pathway may have a cross section that is not circular or complicated inside diameter profile. The geometric factor can be determined by measuring the flow rate (Q) at given pressure (ΔP) with known viscosity (μ) fluid:

$${G}_{f} = \frac{πΔ P}{128 μ Q}$$

The G_{f} value of the optimized fluid pathway may be selected to reduce the max shear stress for each catheter gauge to be the same or less than the max shear stress of a BD 21G VACUTAINER® UltraTouch™ push button blood collection set, which was previously considered the gold standard for blood draws. In some embodiments, G_{f} value of the optimized fluid pathway may be selected to reduce the max shear stress for each catheter gauge to be the same or less than the max shear stress of a BD 25G VACUTAINER® UltraTouch™ push button blood collection set.

In some embodiments, and by way of non-limiting example, an optimized fluid pathway can have a diameter of approximately 0.015 in., approximately 0.020 in., approximately 0.025 in., approximately 0.030 in., approximately 0.035 in., or approximately 0.040 in. In another non-limiting example, a cross-sectional area of an optimized fluid pathway is approximately 0.000152 inch².

The fluid connector devices and associated blood collection systems of the various embodiments described herein additionally provide further advantages over currently existing blood collection systems. For example, add-on fluid connector devices described herein allow for hemolysis-reduction function to be integrated for PIVC blood draw. Further, the fluid connector devices described herein are compatible with PIVC placement and allow for seamless blood draw at insertion. Furthermore, the fluid connector devices have the potential to stay inline throughout PIVC indwell for multiple blood draws. Additionally, since the fluid connector devices are an add-on which can be easily incorporated without any changes to existing PIVC, there is minimal impact to clinical setting and operations.

In some embodiments, the catheter assembly may include or correspond to any suitable catheter assembly. In some embodiments, the catheter assembly may be integrated and include an extension tube, which may extend from and be integrated with a side port of the catheter hub. A non-limiting example of an integrated catheter assembly is the BD NEXIVA™ Closed IV Catheter system, available from Becton Dickinson and Company. In some embodiments, a proximal end of the extension tube may be coupled to an adapter, such as, for example, a Y-adapter or single port luer adapter. In some embodiments, the distal connector of fluid connector device may be configured to couple to the Y-adapter.

In some embodiments, the catheter assembly may be non-integrated and may not include the extension tube. In these and other embodiments, the fluid connector device may be configured to couple to the proximal end of the catheter hub or another suitable portion of the catheter assembly. In some embodiments, the catheter assembly may be coupled to a removable extension tube. In some embodiments, the fluid connector device may be coupled directly to the catheter adapter, eliminating the extension tube and providing a compact catheter system.

Various examples of aspects of the disclosure are described as numbered clauses (1, 2, 3, etc.) for convenience. These are provided as examples and do not limit the subject technology. Identification of the figures and reference numbers are provided below merely as examples for illustrative purposes, and the clauses are not limited by those identifications.

Clause 1: A fluid connector device comprising a first connector, a second connector that combines with the first connector, forming a housing, and a flow restrictor insert with an exterior flow channel, wherein the insert is fitted inside the housing.

Clause 2: The fluid connector device of clause 1, wherein the exterior flow channel comprises a groove extending from a proximal end of the flow restrictor insert to a distal end of flow restrictor insert.

Clause 3: The fluid connector device of clause 2, wherein the groove is a semi-parabolic cylinder.

Clause 4: The fluid connector device of clause 1, wherein the first connector is a female luer.

Clause 5: The fluid connector device of clause 1, wherein the second connector is a male luer.

Clause 6: The fluid connector device of clause 1, wherein the exterior flow channel has a diameter of about 0.015 in., about 0.020 in., about 0.025 in., about 0.030 in., about 0.035 in., or about 0.040 in.

Clause 7: The fluid connector device of clause 1, wherein the exterior flow channel has a length of about 0.70 in., about 0.80 in., about 0.90 in., about 1.0 in., about 1.10 in., about 1.20 in., or about 1.30 in.

Clause 8: The fluid connector device of clause 2, wherein the flow restrictor insert has a maximum diameter of about 0.1 in., about 0.2 in., about 0.3 in., about 0.4 in., about 0.5 in., about 0.6 in., or about 0.7 in.

Clause 9: The fluid connector device of clause 1, wherein the first connector has an inner diameter of about 0.1 in., about 0.2 in., about 0.3 in., about 0.4 in., about 0.5 in., about 0.6 in., or about 0.7 in.

Clause 10: The fluid connector device of clause 1, wherein the second connector has an inner diameter of about 0.1 in., about 0.2 in., about 0.3 in., about 0.4 in., about 0.5 in., about 0.6 in., or about 0.7 in.

Clause 11: The fluid connector device of clause 1, wherein a fluid flows through the exterior flow channel from the first connector towards the second connector.

Clause 12: The fluid connector device of clause 11, wherein the fluid is blood.

Clause 13: The fluid connector device of clause 1, wherein the flow restrictor insert reduces hemolysis during blood draw.

Clause 14: The fluid connector device of clause 1, wherein the fluid connector device is configured to couple to a catheter assembly.

Clause 15: The fluid connector device of clause 1, wherein the flow restrictor insert is contained within the first connector and the second connector.

Clause 16: The fluid connector device of clause 1, wherein the flow restrictor insert is a solid cylinder with the exterior flow channel carved out of an exterior surface of the flow restrictor insert.

Clause 17: A blood collection system comprising a blood collection device, and a fluid connector device fluidly coupled to the blood collection device, the fluid connector device comprising a first connector, a second connector that combines with the first connector, forming a housing, and a flow restrictor insert with an exterior flow channel, wherein the insert is fitted inside the housing.

Clause 18: A method for manufacturing a fluid connector device comprising obtaining a first connector, inserting a flow restrictor insert with an exterior flow channel inside the first connector, obtaining a second connector, and coupling the first connector to the second connector forming a housing; wherein the flow restrictor insert is press fitted inside the housing.

Clause 19: The method of clause 17 further comprising forming the flow restrictor insert into a solid cylinder with a groove carved out of the external surface.

Clause 20: The method of clause 17 further comprising ultrasonically welding the first connector to the second connector.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector device comprising:
a first connector;
a second connector that combines with the first connector, forming a housing; and
a flow restrictor insert with an exterior flow channel, wherein the insert is fitted inside the housing.

2. The fluid connector device of claim 1, wherein the exterior flow channel comprises a groove extending from a proximal end of the flow restrictor insert to a distal end of flow restrictor insert.

3. The fluid connector device of claim 2, wherein the groove is a semi-parabolic cylinder.

4. The fluid connector device of claim 1, wherein the first connector is a female luer.

5. The fluid connector device of claim 1, wherein the second connector is a male luer.

6. The fluid connector device of claim 1, wherein the exterior flow channel has a diameter of about 0.015 in., about 0.020 in., about 0.025 in., about 0.030 in., about 0.035 in., or about 0.040 in.

7. The fluid connector device of claim 1, wherein the exterior flow channel has a length of about 0.70 in., about 0.80 in., about 0.90 in., about 1.0 in., about 1.10 in., about 1.20 in., or about 1.30 in.

8. The fluid connector device of claim 2, wherein the flow restrictor insert has a maximum diameter of about 0.1 in., about 0.2 in., about 0.3 in., about 0.4 in., about 0.5 in., about 0.6 in., or about 0.7 in.

9. The fluid connector device of claim 1, wherein the first connector has an inner diameter of about 0.1 in., about 0.2 in., about 0.3 in., about 0.4 in., about 0.5 in., about 0.6 in., or about 0.7 in.

10. The fluid connector device of claim 1, wherein the second connector has an inner diameter of about 0.1 in., about 0.2 in., about 0.3 in., about 0.4 in., about 0.5 in., about 0.6 in., or about 0.7 in.

11. The fluid connector device of claim 1, wherein a fluid flows through the exterior flow channel from the first connector towards the second connector.

12. The fluid connector device of claim 11, wherein the fluid is blood.

13. The fluid connector device of claim 1, wherein the flow restrictor insert reduces hemolysis during blood draw.

14. The fluid connector device of claim 1, wherein the fluid connector device is configured to couple to a catheter assembly.

15. The fluid connector device of claim 1, wherein the flow restrictor insert is contained within the first connector and the second connector.

16. The fluid connector device of claim 1, wherein the flow restrictor insert is a solid cylinder with the exterior flow channel carved out of an exterior surface of the flow restrictor insert.

17. A blood collection system comprising:
a blood collection device; and
a fluid connector device fluidly coupled to the blood collection device, the fluid connector device comprising:
a first connector;
a second connector that combines with the first connector, forming a housing; and
a flow restrictor insert with an exterior flow channel, wherein the insert is fitted inside the housing.

18. A method for manufacturing a fluid connector device comprising:
obtaining a first connector;
inserting a flow restrictor insert with an exterior flow channel inside the first connector;
obtaining a second connector; and
coupling the first connector to the second connector forming a housing; wherein the flow restrictor insert is press fitted inside the housing.

19. The method of claim 17 further comprising forming the flow restrictor insert into a solid cylinder with a groove carved out of the external surface.

20. The method of claim 17 further comprising ultrasonically welding the first connector to the second connector.
